(19) United States
(12) Patent Application Publication
(10) Pub. No.: US 2015/0119716 A1
(43) Pub. Date: Apr. 30, 2015

(54) METHOD OF ULTRASOUND NONLINEAR IMAGING WITH GOLAY CODE EXCITATION

(71) Applicant: National Taiwan University, Taipei (TW)

(72) Inventor: CHE-CHOU SHEN, TAIPEI (TW)

(73) Assignee: National Taiwan University, Taipei (TW)

(21) Appl. No.: 14/197,326

(22) Filed: Mar. 5, 2014

(30) Foreign Application Priority Data
Oct. 28, 2013 (TW) 102138886

(51) Int. Cl. A61B 8/08

(52) U.S. Cl. CPC A61B 8/5207
USPC 600/447

(57) ABSTRACT

A method of ultrasound nonlinear imaging with Golay code excitation includes transmitting a first and a second Golay code signal wave which are orthogonal by each other; making the second Golay code signal wave be subtracted from the first Golay code signal wave to eliminate a first and a second noise interference wave to generate a third Golay code signal wave; using a first and a second compression filter to process the third Golay code signal wave to generate a first and a second compressed code signal wave; taking the difference between the first compressed code signal wave and the second compressed code signal wave to generate a third compressed code signal wave which includes at least two second-order harmonic waves; processing ultrasound nonlinear imaging by using the multi-frequency harmonic component waves to generate an ultrasound image.

### FIELD OF THE INVENTION

The present invention relates to a method of ultrasound nonlinear imaging with Golay code excitation, and more particularly to a method eliminating interference of neighboring frequency bands using orthogonal Golay code signal waves to enhance resolution and processing ultrasound nonlinear imaging using multi-frequency components.

### BACKGROUND OF THE INVENTION

Traditional ultrasound imaging method adopts linear scattered fundamental signal to image. However, the fundamental signal is susceptible to phase aberration due to the presence of fat layer in the shallow tissue or skin and will result in low imaging quality. As the sound wave is traveling in human tissues during the imaging process, the wave signal will have finite amplitude distortion or generate harmonic signals when encountering strong nonlinear medium such as microbubble contrast agents. When processing tissue imaging, because the magnitude of harmonic signals is lower than the fundamental signal in the beginning, the scattered harmonic signal will suffer from less phase aberration when penetrating the shallow tissue. Thus, tissue harmonic imaging can provide better contrast resolution because it is less susceptible to phase aberration and thus is broadly used in clinical diagnosis.

The contrast agents being used for harmonic imaging are composed of microbubbles. These small bubbles will have harmonic oscillation to generate lots of strong harmonic signals back to the probe when being excited by sound waves. Clinically, the contrast agents are injected into the blood vessel such that the blood vessel would be filled with micro bubbles to strengthen harmonic signals so as to generate a clearer image of blood vessel structure and blood perfusion. That is, the image contrast is enhanced.

A major difference between ultrasound fundamental signal and harmonic signal is the frequency range of echo signal. If the central frequency of the ultrasound signal travelling into the human body is f₀, the imaging method using the frequency signal f₀ of the echo signal is called fundamental imaging, but the imaging method using the harmonic signals with higher frequency, such as 2f₀, 3f₀, is called harmonic imaging. Because these harmonic signals are originated from the nonlinear reaction of the medium to the emitted ultrasound signal, harmonic imaging can be also regarded as nonlinear imaging. As mentioned, it is understood that by using a low frequency filter or high frequency filter to select the frequency range to be received, it is capable to decide whether fundamental imaging or harmonic imaging is performed. The discussion focuses on the analysis of components of second harmonic signal because the second harmonic signal is the strongest one among the various harmonic signals.

Although harmonic imaging is of great importance in clinical diagnosis due to better imaging quality, its weak signal intensity is a major drawback and may significantly degrade imaging sensitivity and penetration. Generally speaking, harmonic signal can be at least 20 db weaker than the fundamental signal even at the focus. Thus, there have been some researches and inventions focusing on using code excitation to enhance harmonic wave intensity. Among the various coding technologies, Golay code is easy to use and is quite applicable to code excitation. Golay code is performed by phase coded sequence. That is, the emitted signal has the phase 0° is represented by the symbol [1], the emitted signal has the phase 90° is represented by the symbol [j], the emitted signal has the phase 180° is represented by the symbol [−1], and the emitted signal has the phase 270° is represented by the symbol [−j]. Golay code featuring phase coding can be easily implemented on the hardware. However, Golay code excitation needs two emitting processes A and B to generate the corresponding echo signals complementary to each other. That is, the autocorrelation results of the two echo signals can be summed to totally remove the sidelobe interference.

Multi-frequency excitation has been developed in ultrasound nonlinear imaging. The feature of multi-frequency excitation is to emit multiple frequency components rather than single frequency component. If only considering the second-order nonlinear components, the ultrasound nonlinear signals generated by multi-frequency excitation will include the second harmonic signals of each emitting frequencies and the inter-modulation signal between the emitting frequencies. Thus, in addition to the second harmonic signals being used in typical harmonic imaging, the inter-modulation signals can also be used for generating image. However, multi-frequency excitation using Golay code excitation for imaging will result in incorrect coding of some harmonic wave components, such as second-order harmonic wave and fourth-order harmonic wave. These incorrectly coded components will interfere with the correctly coded signals to cause degradation in imaging quality.

Take nonlinear imaging using two-bit dual-frequency Golay excitation as an example, it is capable to have the frequency components f₂−f₁ and 2f₁ of the second-order harmonic wave showing the correct code [1, −1] during emission A and the correct code [−1, −1] during emission B as shown in the following table. The above mentioned frequency components of the second-order harmonic wave are usually within the pass band of the probe and thus serve as the major signal components for imaging.

| | Transmit | | 2^{nd}-order Harmonic | | | 4^{th}-order Harmonic | | | | |
|---|---|---|---|---|---|---|---|---|---|---|
| Frequency | f₁ | f₂ | f₂ − f₁ | 2f₁ | f₂ + f₁ | f₂ − f₁ | | 2f₁ | | f₂ + f₁ |
| Golay code | [1, j] | [1, −j] | [1, −1] | [1, −1] | [1, 1] | [1, 1] | [1, −1] | [1, −1] | [1, 1] | [1, 1] |
| (Emission A) | | | | | | | | | | |
| Golay code | [j, j] | [−j, −j] | [−1, −1] | [−1, −1] | [1, 1] | [1, 1] | [−1, −1] | [−1, −1] | [1, 1] | [1, 1] |
| (Emission B) | | | | | | | | | | |

However, as shown in this table, it is understood that among the other harmonic wave components, component f₂+f₁ of the second-order harmonic wave and components f₂−f₁ and 2f₁ of the fourth-order harmonic wave may not accord with the designed Golay code. The codes are all [1, 1]. These frequency components with incorrect code will result in unremovable sidelobe signals during the compression process and the method nowadays cannot effectively resolve this problem.

### BRIEF SUMMARY OF INVENTION

As mentioned, when using Golay code excitation in multi-frequency harmonic imaging, it is demanded to: (1) enhance signal-to-noise ratio (SNR); (2) prevent the correctly coded signals from being interfered by the incorrectly coded components. However, the method provided in the publications nowadays can only achieve the first request but fail to disclose a concrete method to achieve both the two above mentioned requests when using Golay code excitation in multi-frequency harmonic imaging.

Accordingly, it is an object of the present invention to provide a method of ultrasound nonlinear imaging with Golay code excitation, which emits signals with two sets of Golay code signals orthogonal with each other, removes the interference of the incorrect coded components within the two sets of signals, and compresses the signals to generate the wave including multi-frequency components for imaging.

Based on the above mentioned object, a method of ultrasound nonlinear imaging with Golay code excitation is provided in accordance with an embodiment of the present invention, which comprises the steps of: (a) receiving a first Golay code signal wave and a second Golay code signal wave, wherein the first Golay code signal wave includes at least two first second-order harmonic waves and at least one first noise interference wave, the second Golay code signal wave includes at least two second second-order harmonic waves and at least one second noise interference wave, the above mentioned at least two first second-order harmonic wave are encoded by a first code signal, and the at least two second second-order harmonic waves are encoded by a second code signal, and the first code signal and the second code signal are orthogonal by each other; (b) making the second Golay code signal wave be subtracted from the first Golay code signal wave to eliminate the first and the second noise interference wave to generate a third Golay code signal wave; (c) performing a first and a second compression filter process to the third Golay code signal wave to generate a first and a second compressed code signal wave respectively; (d) taking the difference between the first compressed code signal wave and the second compressed code signal wave to generate a third compressed code signal wave which includes at least two compressed second-order harmonic waves; and (e) processing ultrasound nonlinear imaging by using the at least two compressed second-order harmonic waves to generate an ultrasound image.

As a preferred embodiment of the present invention, in step (a), the first noise interference wave and the second noise interference wave have identical code. In addition, in step (a), the first code signal and the second code signal are binary code signals, and in step (b), the third Golay code signal includes at least two third second-order harmonic waves, which is generated by subtracting the at least two second second-order harmonic waves from the at least two first second-order harmonic waves. Moreover, in step (c), the first compression filter process is performed by using operation of cross-correlation of the third Golay code signal wave and the first code signal, and the second compression filter process is performed by using operation of cross-correlation of the third Golay code signal wave and the second code signal.

The method of ultrasound nonlinear imaging with Golay code excitation provided in accordance with the present invention removes the noise signals before the compression filtering such that the Golay code can be correctly decoded. Thus, the Golay code waveform provided in the present invention is capable to not only enhance SNR but also resolve the interference from the incorrectly coded components so as to enhance imaging quality.

The embodiments adopted in the present invention would be further discussed by using the following paragraph and the figures for a better understanding.

### BRIEF DESCRIPTION OF THE DRAWINGS

FIG. 1 is a flow chart showing a method of ultrasound nonlinear imaging with Golay code excitation in accordance with a preferred embodiment of the present invention.

FIG. 2 is a schematic view showing the waveform of the first Golay code signal, the second Golay code signal, and the third Golay code signal in accordance with a preferred embodiment of the present invention.

FIG. 3 is a schematic view showing the generation of the first compressed code signal waveform and the second compressed code signal waveform from the third Golay code signal waveform in accordance with a preferred embodiment of the present invention.

FIG. 4 is a schematic view showing the generation of the third compressed code signal waveform from the first compressed code signal waveform and the second compressed code signal waveform in accordance with a preferred embodiment of the present invention.

FIG. 5 is a first diagram showing the effect of interference removal with a preferred embodiment of the present invention.

FIG. 5A is a second diagram showing the effect of interference removal with a preferred embodiment of the present invention.

### DETAILED DESCRIPTION OF THE INVENTION

There are various embodiments of the method of ultrasound nonlinear imaging with Golay code excitation in accordance with the present invention, which are not repeated hereby. The preferred embodiments are mentioned in the following paragraph as an example. It should be understood by those skilled in the art that the preferred embodiments disclosed in the following paragraph are merely an example instead of restricting the scope of the invention itself.

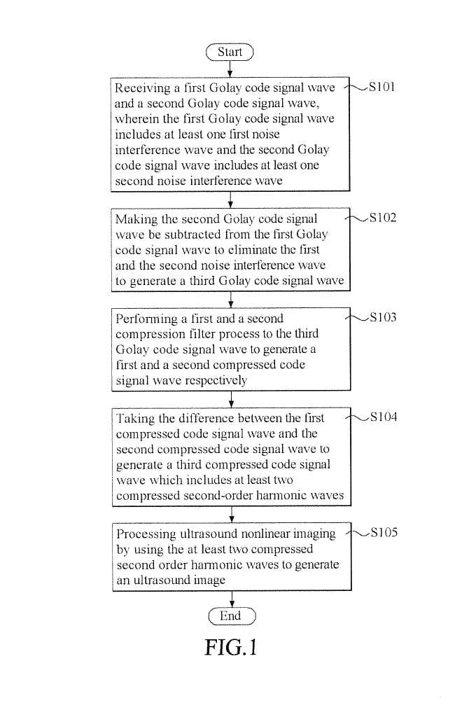

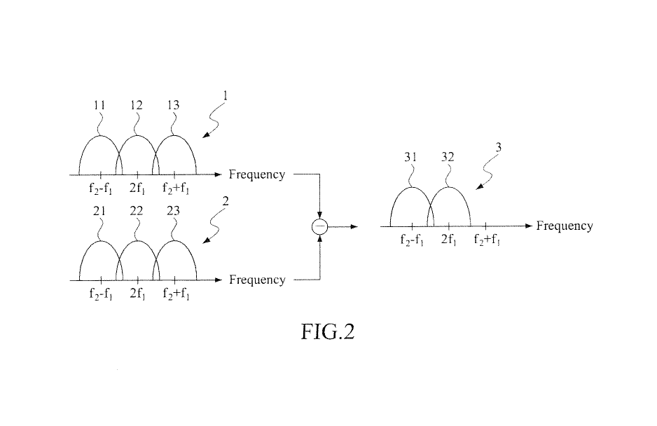

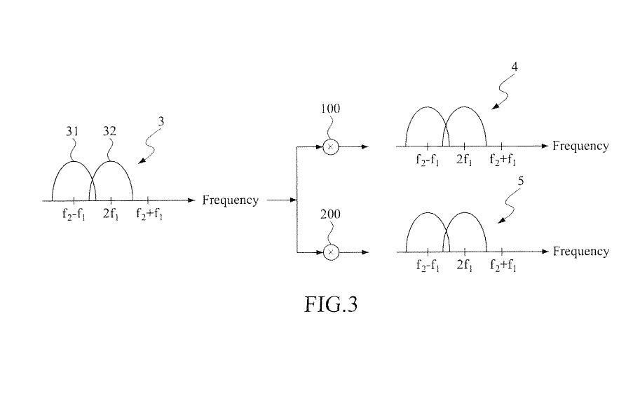

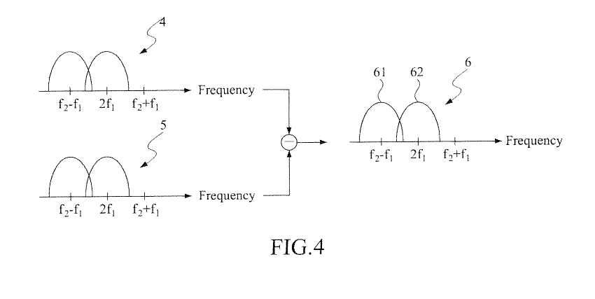

FIG. 1 is a flow chart showing a method of ultrasound nonlinear imaging with Golay code excitation in accordance with a preferred embodiment of the present invention, FIG. 2 is a schematic view showing the waveform of the first Golay code signal, the second Golay code signal, and the third Golay code signal in accordance with a preferred embodiment of the present invention, FIG. 3 is a schematic view showing the generation of the first compressed code signal waveform and the second compressed code signal waveform from the third Golay code signal waveform in accordance with a preferred embodiment of the present invention, and FIG. 4 is a schematic view showing the generation of the third compressed code signal waveform from the first compressed code signal waveform and the second compressed code signal waveform in accordance with a preferred embodiment of the present invention;

As shown, the method of ultrasound nonlinear imaging with Golay code excitation provided in accordance with an embodiment of the present invention comprises the steps of:

Step S101: receiving a first Golay code signal wave and a second Golay code signal wave, wherein the first Golay code signal wave includes at least one first noise interference wave, the second Golay code signal wave includes at least one second noise interference wave;

Step S102: making the second Golay code signal wave be subtracted from the first Golay code signal wave to eliminate the first and the second noise interference wave to generate a third Golay code signal wave;

Step S103: performing a first and a second compression filter process to the third Golay code signal wave to generate a first and a second compressed code signal wave respectively;

Step S104: taking the difference between the first compressed code signal wave and the second compressed code signal wave to generate a third compressed code signal wave which includes at least two compressed second-order harmonic waves; and

Step S105: processing ultrasound nonlinear imaging by using the at least two compressed second-order harmonic waves to generate an ultrasound image.

After the process starts, step S101 is carried out to receive the first Golay code signal wave and the second Golay code signal wave, wherein the first Golay code signal wave includes at least one first noise interference wave and the second Golay code signal wave includes at least one second noise interference wave. As shown in FIG. 2, two phase coding waves with frequency f₁ and f₂ (f₂ is greater than f₁) are emitted before the present step, the first Golay code signal wave 1 and the second Golay code signal wave 2 are received through adjusting the phase. The first Golay code signal wave 1 includes two first second-order harmonic waves 11, 12 (in the other embodiments, the Golay code signal wave may include more than two harmonic waves) and at least one first noise interference wave 13 (in the other embodiments, the Golay code signal wave may include more than one noise interference wave). The first second-order harmonic waves 11, 12 are with respective to a first code signal (not shown). As a preferred embodiment of the present invention, the first code signal is binary coded, such as [1, −1]. In addition, the first second-order harmonic waves 11, 12 are the waves with correct code.

The first noise interference wave 13 has a corresponding code signal, which is also a binary code signal, such as [1,1]. It is noted that the first noise interference wave 13 is the wave of the incorrectly coded interference signal as mentioned in the prior art. The first noise interference wave 13 is partially overlapped with the first second-order harmonic wave 12. The central frequency of the first second-order harmonic wave 11 is f₂−f₁, the central frequency of the first second-order harmonic wave 12 is 2f₁, and the central frequency of the first noise interference wave 13 is f₂+f₁.

The second Golay code signal wave 2 includes two second second-order harmonic waves 21, 22 (in other embodiments, the Golay code signal wave may include more than two harmonic waves) and at least one second noise interference wave 23 (in other embodiments, the Golay code signal wave may include more than one noise interference wave). The second second-order harmonic waves 21, 22 are with respective to a second code signal (not shown). As a preferred embodiment, the second code signal is binary coded, such as [−1,−1]. In addition, the second second-order harmonic waves 21,22 are the waves with correct code. The second noise interference wave 23 has a corresponding code signal (not shown), which is identical to that of the first noise interference wave 13, both are [1,1]. The second noise interference wave 23 is also the interference wave with incorrect code as mentioned in the prior art. The second noise interference wave 23 is partially overlapped with the second second-order harmonic wave 22.

It is noted that in accordance with a preferred embodiment of the present invention, the first code signal and the second code signal are orthogonal with each other. The definition of orthogonal in the present invention is that the sidelobe signal can be removed by summing the cross-correlation compression results of the first code signal and the second code signal. For example, if A is the first code signal, B is the second code signal, and the sum of cross-correlation compression result of A and B and cross-correlation compression result of B and A is 0, then the two code signals A and B are orthogonal with each other. If the sum of auto-correlation compression result of A and A and auto-correlation compression result of B and B is δ, the two code signals A and B are complementary with each other. Cross-correlation compression and auto-correlation compression are well known technologies and thus are not repeated here. In addition, the central frequency of the second second-order harmonic wave 21 is f₂−f₁, the central frequency of the second second-order harmonic wave 22 is 2f₁, and the central frequency of the second noise interference wave 23 is f₂+f₁.

After the completion of step S 101, step S102 is carried out to make the second Golay code signal wave 2 be subtracted from the first Golay code signal wave 1 so as to generate a third Golay code signal wave 3. In detail, as shown in FIG. 2, the noise interference with incorrect code is removed in this step. As a preferred embodiment, a subtractor (not shown in the figure) is used to make the second Golay code signal wave 2 be subtracted from the first Golay code signal wave 1 to eliminate the first noise interference wave 13 and the second noise interference wave 23 so as to generate the third Golay code signal wave 3. The third Golay code signal wave 3 includes two third second-order harmonic waves 31,32, wherein the third second-order harmonic wave 31 is generated by subtracting the second second-order harmonic wave 21 from the first second-order harmonic wave 11, and the third second-order harmonic wave 32 is generated by subtracting the second second-order harmonic wave 22 from the first second-order harmonic wave 12.

The central frequency of the third second-order harmonic wave 31 is f₂−f₁ and the corresponding code is [2,0], and the central frequency of the third second-order harmonic wave 32 is 2f₁ and the corresponding code is also [2,0].

After the completion of step S102, step S103 is performed to process a first and a second compression filter process to the third Golay code signal wave 3 so as to generate a first and a second compressed code signal wave respectively. Specifically, as shown in FIG. 3, the first compression filter process 100 and the second compression filter process 200 are carried out on the third Golay code signal wave 3 simultaneously. As a preferred embodiment, the first compression filter process 100 is to process cross-correlation compression on the third Golay code signal wave 3 and the first code signal, e.g. [1,−1] in the present embodiment, by using a filter, and the second compression filter process 200 is to process cross-correlation compression on the third Golay code signal wave 3 and the second code signal, e.g. [−1,−1] in the present embodiment, by using a filter.

After the completion of processing the first compression filter process 100 to the third Golay code signal wave 3, a first compressed code signal wave 4 is generated, which also includes the second-order harmonic wave (not labeled in the figure), and after the completion of processing the second compression filter process 200 to the third Golay code signal wave 3, a second compressed code signal wave 5 is generated, which also includes the second-order harmonic wave (not labeled in the figure). However, because the first compressed code signal wave 4 and the second compressed code signal wave 5 generated from the step S103 are not fully decoded, it is demanded to perform the step S 104.

After the completion of step S 103, S104 is carried out to take the difference between the first compressed code signal wave and the second compressed code signal wave so as to generate a third compressed code signal wave which includes at least two compressed second-order harmonic waves. As a preferred embodiment, the present step may use a subtractor to subtract the second compressed code signal wave 5 from the first compressed code signal wave 4 so as to generate the fully decoded third compressed code signal wave 6, which includes two compressed second-order harmonic waves 61, 62 in the present embodiment. The third compressed code signal wave 6 may include more than two second-order harmonic waves in other embodiments. The central frequency of the compressed second-order harmonic wave 61 is f₂−f₁ and the corresponding code is [0,4,0], and the central frequency of the compressed second-order harmonic wave 62 is 2f₁ and the corresponding code is also [0,4,0].

After the completion of step S 104, step S105 is carried out to process ultrasound nonlinear imaging by using the compressed second-order harmonic waves so as to generate an ultrasound image. Specifically, the compressed second-order harmonic wave 61 and the compressed second-order harmonic wave 62 are used in the present step to process ultrasound nonlinear imaging. Ultrasound imaging is well known to the person skilled in the art and thus is not repeated here. By using the above mentioned steps, image resolution along the axial direction can be remained so as to generate a clearer ultrasound image.

In addition, the technology disclosed in the present invention can be applied to nonlinear imaging or the conditions that the fundamental signal being interfered by the second-order harmonic waves or the second-order harmonic wave being interfered by the fourth-order harmonic wave. Thus, the application of the present invention should not be restricted in nonlinear imaging.

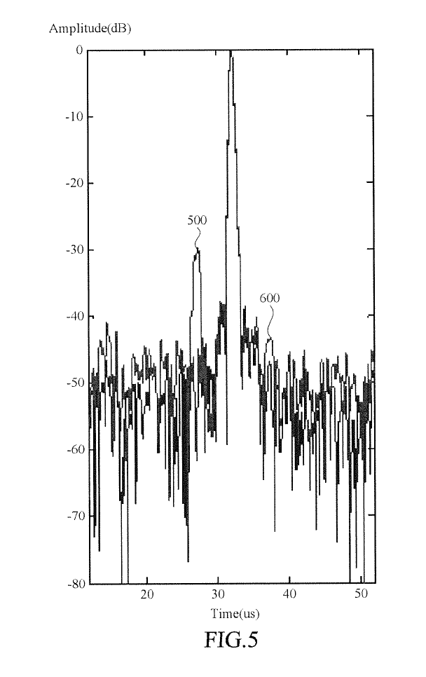

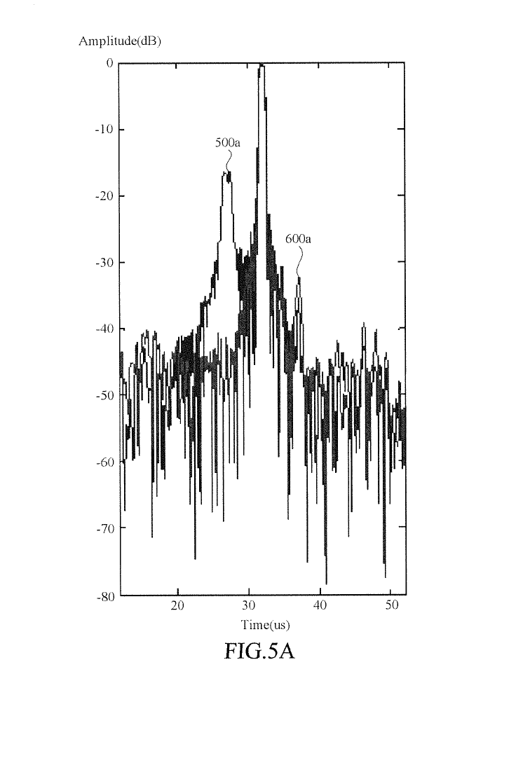

FIGS. 5 and 5A are the diagrams showing the effect to suppress sidelobe signals. FIG. 5 shows the signal envelope at f₂−f₁ frequency, and FIG. 5A shows the signal envelope at 2f₁ frequency. Specifically, waves 500 and 500a are those using the original Golay code, and the waves 600 and 600a are those using the technology of the present invention. As shown, significant sidelobe signals can be found near the front end of the mainlobe signal on the waves 500 and 500a. These sidelobe signals result from the interferences with incorrect code during compression. In contrast, as shown in the waves 600 and 600a, by using the technology provided in the present invention, the enormous sidelobe signals before the mainlobe signal have been totally suppressed and the width of the mainlobe signal can be remained to prevent image resolution along the axial direction from being degraded.

In conclusion, the method of ultrasound nonlinear imaging with Golay code excitation provided in accordance with the present invention removes the interference signals before processing the compression process such that the Golay code can be correctly decoded. Thus, the Golay code waveform provided in the present invention is capable to not only enhance SNR but also eliminate the interference with incorrect code so as to enhance imaging quality.

The detail description of the aforementioned preferred embodiments is for clarifying the feature and the spirit of the present invention. The present invention should not be limited by any of the exemplary embodiments described herein, but should be defined only in accordance with the following claims and their equivalents. Specifically, those skilled in the art should appreciate that they can readily use the disclosed conception and specific embodiments as a basis for designing or modifying other structures for carrying out the same purposes of the present invention without departing from the scope of the invention as defined by the appended claims.

What is claimed is:

1. A method of ultrasound nonlinear imaging with Golay code excitation comprising the steps of:
(a) receiving a first Golay code signal wave and a second Golay code signal wave, wherein the first Golay code signal wave includes at least two first second-order harmonic waves and at least one first noise interference wave, the second Golay code signal wave includes at least two second second-order harmonic waves and at least one second noise interference wave, the above mentioned at least two first second-order harmonic wave are respective to a first code signal, and the at least two second second-order harmonic waves are respective to a second code signal, and the first code signal and the second code signal are orthogonal by each other;
(b) making the second Golay code signal wave be subtracted from the first Golay code signal wave to eliminate the first and the second noise interference wave to generate a third Golay code signal wave;
(c) performing a first and a second compression filter process to the third Golay code signal wave to generate a first and a second compressed code signal wave respectively;
(d) taking the difference between the first compressed code signal wave and the second compressed code signal wave to generate a third compressed code signal wave which includes at least two compressed second-order harmonic waves; and
(e) processing ultrasound nonlinear imaging by using the at least two compressed second-order harmonic waves to generate an ultrasound image.

2. The method of ultrasound nonlinear imaging with Golay code excitation of claim 1, wherein in step (a), the first noise interference wave and the second noise interference wave have identical code.

3. The method of ultrasound nonlinear imaging with Golay code excitation of claim 1, wherein in step (a), the first code signal and the second code signal are binary code signals.

4. The method of ultrasound nonlinear imaging with Golay code excitation of claim 1, wherein in step (b), the third Golay code signal includes at least two third second-order harmonic waves, which is generated by subtracting the at least two second second-order harmonic waves from the at least two first second-order harmonic waves.

5. The method of ultrasound nonlinear imaging with Golay code excitation of claim 1, wherein in step (c), the first compression filter process is performed by using operation of cross-correlation of the third Golay code signal wave and the first code signal, and the second compression filter process is performed by using operation of cross-correlation of the third Golay code signal wave and the second code signal.
